(19) United States
(12) Patent Application Publication
(10) Pub. No.: US 2012/0259177 A1
(43) Pub. Date: Oct. 11, 2012

(54) Overlapping Retractor Blade Assemblies

(75) Inventor: David Fiorella, Memphis, TN (US)

(73) Assignee: WARSAW ORTHOPEDIC, INC., Warsaw, IN (US)

(21) Appl. No.: 13/079,893

(22) Filed: Apr. 5, 2011

(51) Int. Cl. A61B 1/32

(52) U.S. Cl. 600/228

(57) ABSTRACT

A retractor assembly for percutaneous surgery in a patient includes first and second retractor portions positionable opposite one another in an incision of the patient. A working channel extends between the first and second retractor portions and provides access to a location within the patient adjacent to distal ends of the first and second retractor portions. The first and second retractor portions are movable relative to one another to adjust the size of the working channel. In addition, the first and second retractor portions are further configured to longitudinally overlap with one another such that the working channel is peripherally surrounded or enclosed by the first and second retractor portions as the working channel is adjusted across a plurality of different sizes. In another form, a method is directed to retracting tissue for percutaneous access to a surgical site. However, other embodiments, forms and applications are also envisioned.

### BACKGROUND

The present application relates to tissue retraction to facilitate a procedure, such as minimally invasive surgery, within a patient.

Traditional surgical procedures for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. In one form, access to locations within the body is provided by a working channel between oppositely positioned retractor blades. If necessary, the retractor blades can be moved relative to one another to adjust the size of the working channel. However, when the retractor blades are displaced away from one another to enlarge the working channel for example, tissue surrounding the retractor blades can encroach into the working channel and obstruct access to the location within the body. Thus, while developments in minimally invasive surgery are steps in the right direction, there remains a need for further developments in minimally invasive surgical instruments and methods.

### SUMMARY

One nonlimiting embodiment of the present application is directed to a retractor assembly for percutaneous surgery in a patient that includes first and second retractor portions positionable opposite one another in an incision of the patient. A working channel extends between the first and second retractor portions and provides access to a location within the patient adjacent to distal ends of the first and second retractor portions. The first and second retractor portions are movable relative to one another to adjust the size of the working channel. In addition, the first and second retractor portions are further configured to longitudinally overlap with one another such that the working channel is peripherally surrounded or enclosed by the first and second retractor portions as the working channel is adjusted across a plurality of different sizes. However, in other embodiments, different forms and applications are envisioned.

Another embodiment of the present application is a unique apparatus for percutaneous surgery in a patient. Other embodiments include unique methods, systems, devices, kits, assemblies, equipment, and/or apparatus involving a retractor.

Further embodiments, forms, features, aspects, benefits, objects and advantages of the present application shall become apparent from the detailed description and figures provided herewith.

### BRIEF DESCRIPTION OF THE FIGURES

FIG. 1 is a front plan view of retractor assembly in an insertion configuration.

FIG. 2 is a top view of the retractor assembly illustrated in FIG. 1.

FIG. 3 is a bottom view of the retractor assembly illustrated in FIG. 1.

FIG. 4 is a perspective view of a first retractor portion of the retractor assembly illustrated in FIG. 1.

FIG. 5 is a side plan view of the retractor portion illustrated in FIG. 4.

FIG. 6 is a front plan view of the retractor portion illustrated in FIG. 4.

FIG. 7 is a section view of the retractor portion of FIG. 4 taken along view line 7-7 of FIG. 6.

FIG. 8 is a perspective view of a second retractor portion of the retractor assembly illustrated in FIG. 1.

FIG. 9 is a side plan view of the retractor portion illustrated in FIG. 8.

FIG. 10 is a front plan view of the retractor portion illustrated in FIG. 8.

FIG. 11 is a section view of the retractor portion of FIG. 8 taken along view line 11-11 of FIG. 10.

FIG. 12 is a front plan view of the retractor assembly illustrated in FIG. 1 in an expanded configuration.

FIG. 13 is a rear plan view of the retractor assembly illustrated in FIG. 12.

FIG. 14 is a bottom view of the retractor assembly illustrated in FIG. 12.

FIG. 15 is a perspective view of the retractor assembly illustrated in FIG. 12.

FIG. 16 is a top plan view of a separation instrument structured for manipulation of the retractor assembly illustrated in FIG. 1.

FIGS. 17-23 are views of a sequential dilator set for use with the retractor assembly illustrated in FIG. 1.

FIG. 24 is a front, plan view of the retractor assembly illustrated in FIG. 1 positioned relative to the sequential dilator set illustrated in FIGS. 17-23.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices and described methods, and any such further applications of the principles of the invention as illustrated herein are contemplated as would normally occur to one skilled in the art to which the invention relates.

Instruments and methods for performing percutaneous surgery, including spinal surgeries that include one or more techniques such as laminotomy, laminectomy, foramenotomy, facetectomy, discectomy, interbody fusion, spinal nucleus or disc replacement, and implant insertion including plates, rods, and bone engaging fasteners, for example, are provided. The surgery is performed through a working channel or passageway through skin and tissue of the patient provided by a retractor assembly. Viewing of the surgical site at the working end of the retractor assembly can be accomplished with viewing instruments mounted on the retractor assembly, positioned over the retractor assembly, positioned in other portals in the body, and/or through a viewing system such as lateral fluoroscopy. The retractor assembly is movable in situ to increase the size of the working channel to facilitate access to the working space at the distal end of the retractor assembly while minimizing trauma to tissue surrounding the retractor. The retractor can be used with any surgical approach to the spine, including anterior, posterior, posterior mid-line, lateral, postero-lateral, and/or antero-lateral approaches, and in other regions besides the spine.

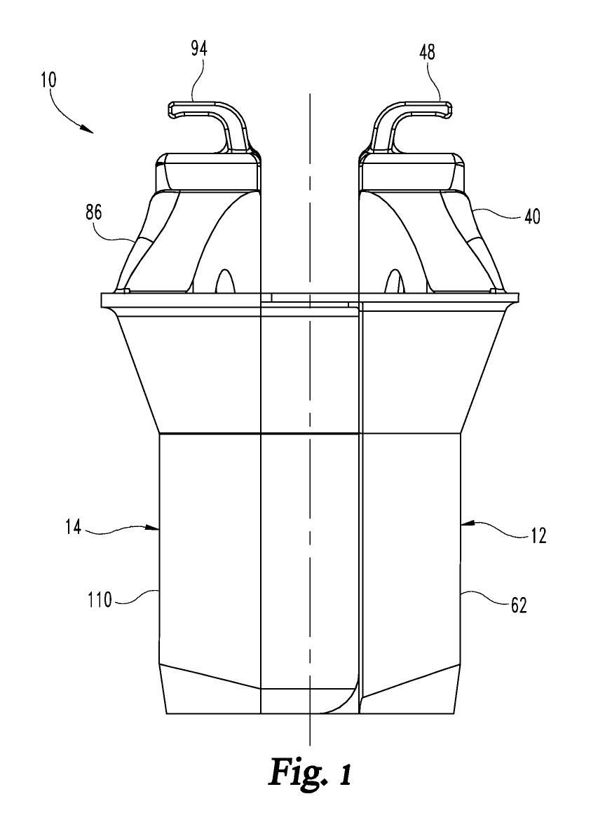

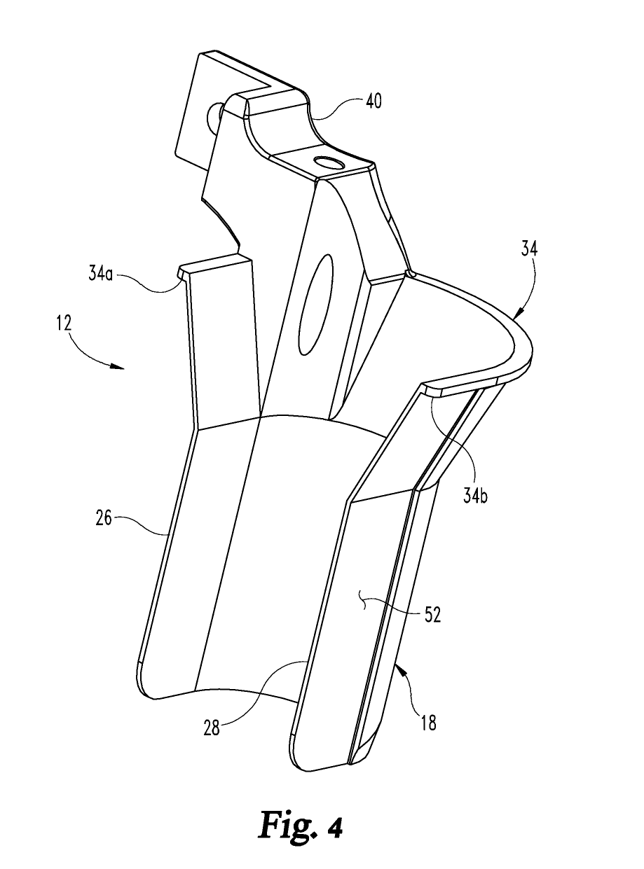

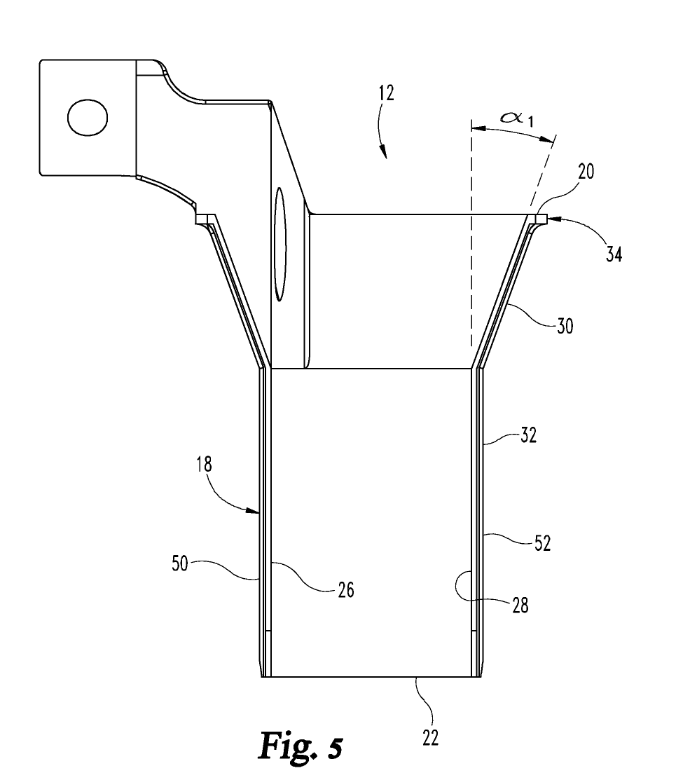

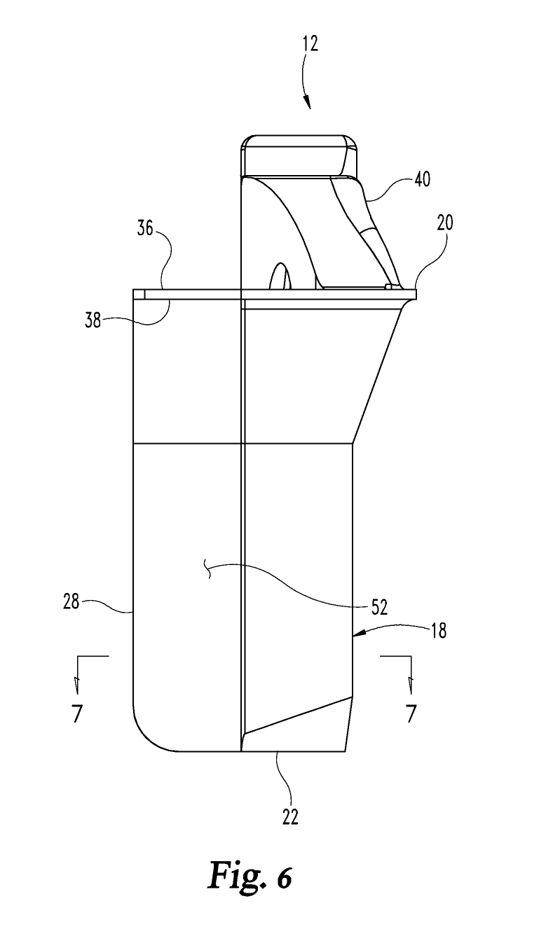

Referring now generally to FIGS. 1-3, there is illustrated one embodiment retractor assembly 10 in an insertion configuration. Assembly 10 includes a first retractor portion 12 positioned opposite of a second retractor portion 14. A working channel 16 is positioned between first and second retractor portions 12, 14. With further reference to FIGS. 4-7, first retractor portion 12 is generally in the form of a retractor blade and includes an elongate body 18 having a sidewall 19 extending between a proximal end 20 and an opposite distal end 22. Sidewall 19 of elongate body 18 further extends between oppositely positioned longitudinal edges 26, 28 which extend between proximal and distal ends 20, 22, and includes a proximal portion 30 obliquely oriented relative to a distal portion 32 at an angle α₁. In one form, angle α₁ is between about 10 and about 30 degrees, although other values for α₁ are contemplated. In other non-illustrated forms, it is contemplated that sidewall 19 can extend linearly between proximal and distal ends 20, 22 without obliquely extending proximal portion 30.

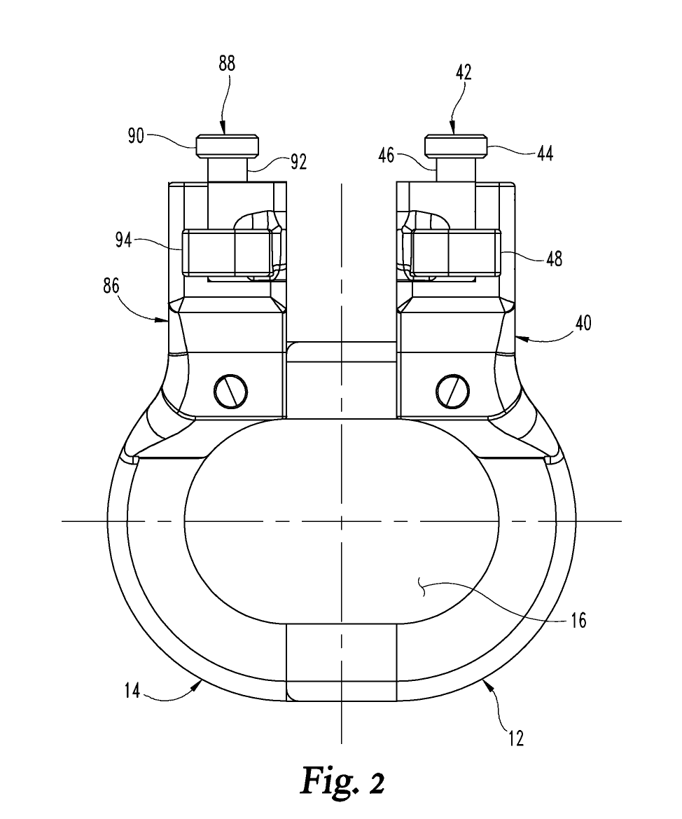

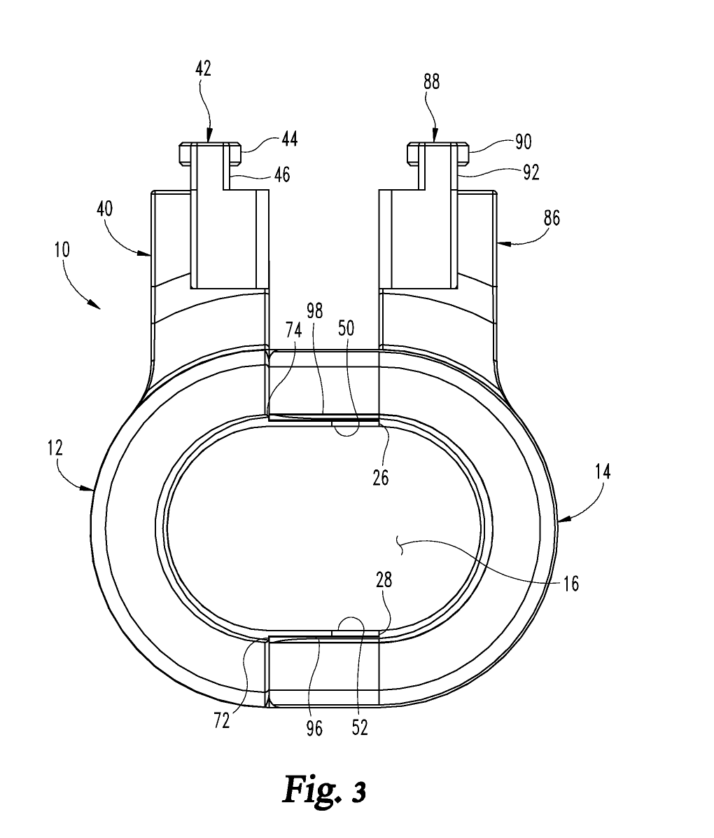

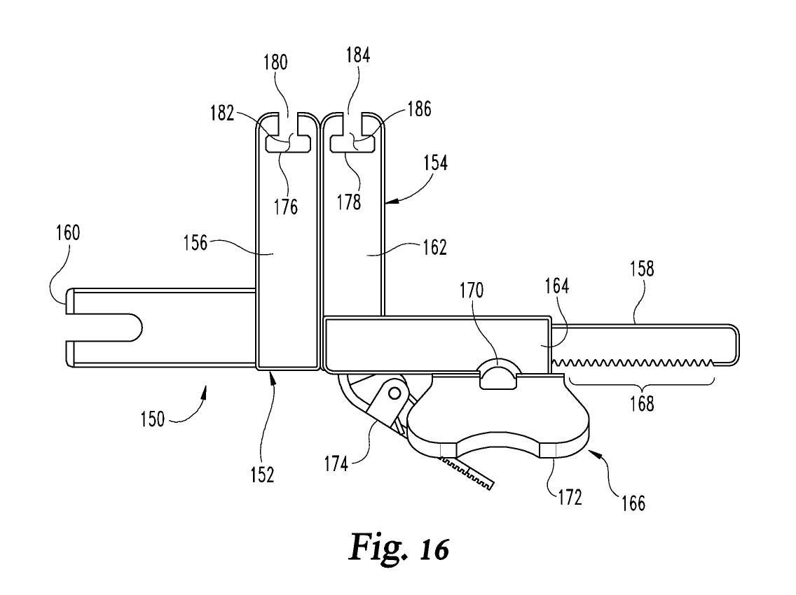

Proximal end 20 includes a rim 34 that extends laterally over proximal portion 30 between opposite medially facing ends 34a, 34b. However, forms in which rim 34 is flush with proximal portion 30 are also contemplated. Rim 34 further includes a proximal facing surface 36 positioned opposite of a distal facing surface 38. Extending proximally and laterally from proximal end 20 of elongate body 18 is a first engagement member 40 including a foot 42 (shown in FIGS. 2 and 3 for example) slidably and removably receivable in a corresponding recess of a separation instrument, further details of which will be provided below in connection with FIG. 16. In the illustrated embodiment, foot 42 includes an enlarged outer portion 44 and a smaller cross-section intermediate transition portion 46. First engagement member 40 also includes a hook portion 48 configured to engage with one or more other surgical instruments, non-limiting examples of which will be provided below, or with an external arm that supports retractor assembly 10 while positioned in the patient. For the sake of clarity, it should be appreciated that foot 42 and hook portion 48 are releasably coupled with first engagement member 40 in the illustrated form and have not been shown in FIGS. 4-7. In addition, it should be appreciated that first engagement member 40 may be releasably or non-releasably coupled with elongate body 18, and in some alternative, non-illustrated forms may be absent from first retractor portion 12. While not previously discussed, it should be appreciated that distal end 22 is slightly beveled to facilitate insertion of first retractor portion 12 into an incision, although non-beveled forms for distal end 22 are also contemplated.

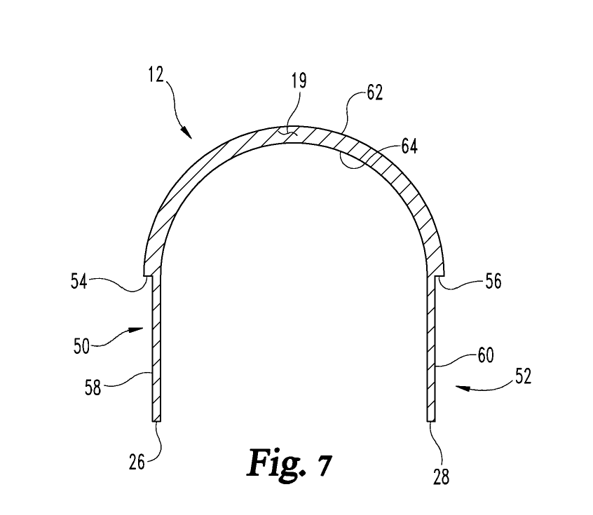

Sidewall 19 of elongate body 18 includes a pair of reduced thickness portions 50, 52 positioned adjacent to longitudinal edges 26, 28, respectively. Similarly, reduced thickness portions 50, 52 define indentations or recessed portions that extend from longitudinal edges 26, 28 to medially facing surfaces 54, 56, respectively. Portions 50, 52 also extend from distal end 22 to distal facing surface 38 of rim 38. Sidewall 19 of elongate body 18 also includes first and second external surface portions 58, 60 extending along reduced thickness portions 50, 52, respectively, and a third external surface portion 62 extending between reduced thickness portions 50, 52. As illustrated in FIG. 7 for example, external surface portions 58, 60 are inwardly offset toward working channel 16 relative to external surface portion 62. In contrast, internal surface 64 extending between longitudinal edges 26, 28 of elongate body 18 is uninterrupted and substantially smooth, although configurations in which internal surface 64 is alternatively arranged are also possible.

While not previously discussed, it should be appreciated that distal portion 32 of sidewall 19 is generally linear in both its longitudinal and lateral aspects along reduced thickness portions 50, 52 such that reduced thickness portions 50, 52 extend substantially parallel to one another along distal portion 32. Moreover, distal portion 32 of sidewall 19 is generally arcuately shaped between reduced thickness portions 50, 52 such that elongate body 18 has a generally u-shaped cross section along distal portion 32 as illustrated in FIG. 7 for example. Moreover, proximal portion 30 of sidewall 19 is generally linear in both its longitudinal and lateral aspects along reduced thickness portions 50, 52, although reduced thickness portions 50, 52 extend transversely to one another along proximal portion 30 given the oblique relationship between proximal and distal portions 30, 32. Moreover, proximal portion 30 of sidewall 19 is generally arcuately shaped between reduced thickness portions 50, 52 such that elongate body 18 also generally has a u-shaped cross section along proximal portion 30. Other cross-sectional shapes are also contemplated for sidewall 19, such as, for example, any open sided polygonal shape or combined curved/polygonal shape, just to provide a few examples.

First retractor portion 12 can be provided with sufficient rigidity between proximal and distal ends 20, 22 to separate and maintain separation of adjacent tissue when first and second retractor portions 12, 14 are initially inserted and also when the adjacent tissue is retracted by moving first retractor portion 12 and second retractor portion 14 away from one another. For example, first retractor portion 12 can include a thickness between reduced thickness portions 50, 52 which provides sufficient rigidity to resist bending or bowing under the forces exerted on it by the retracted tissue and/or muscle. Also, the generally semicircular or u-shaped cross-section of first portion 12 can be configured to provide a sufficient section modulus or moment of inertia in the direction of movement of first retractor portion 12 to resist bending, bowing and/or deflection forces applied during such movement.

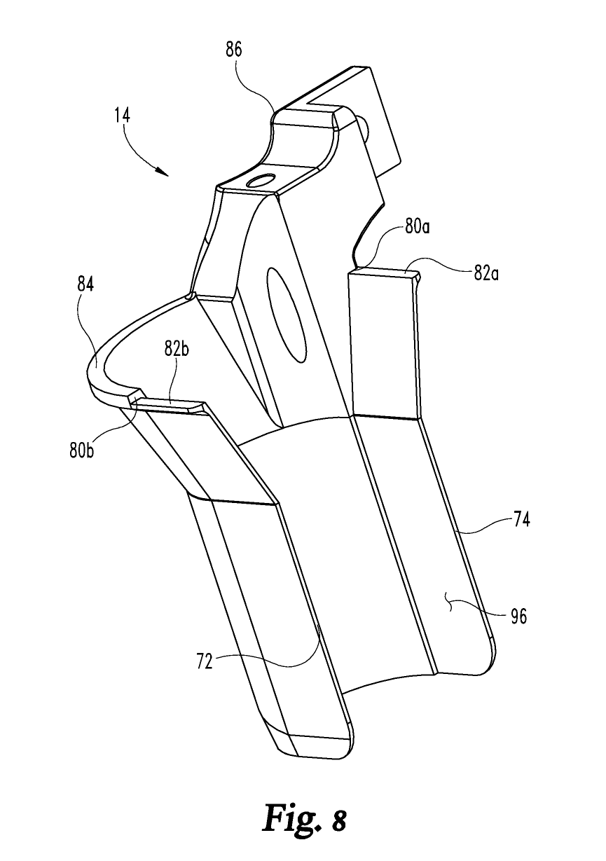

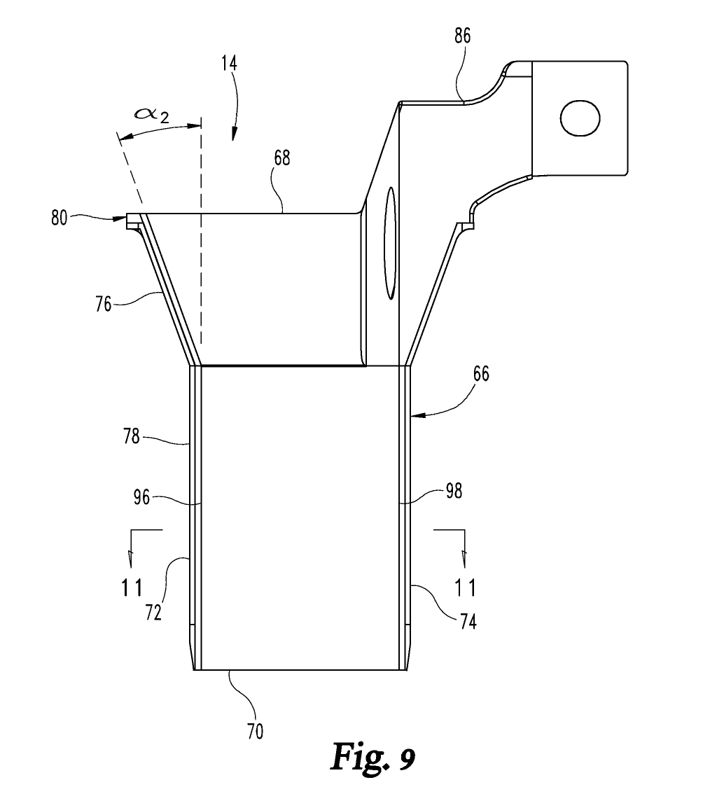

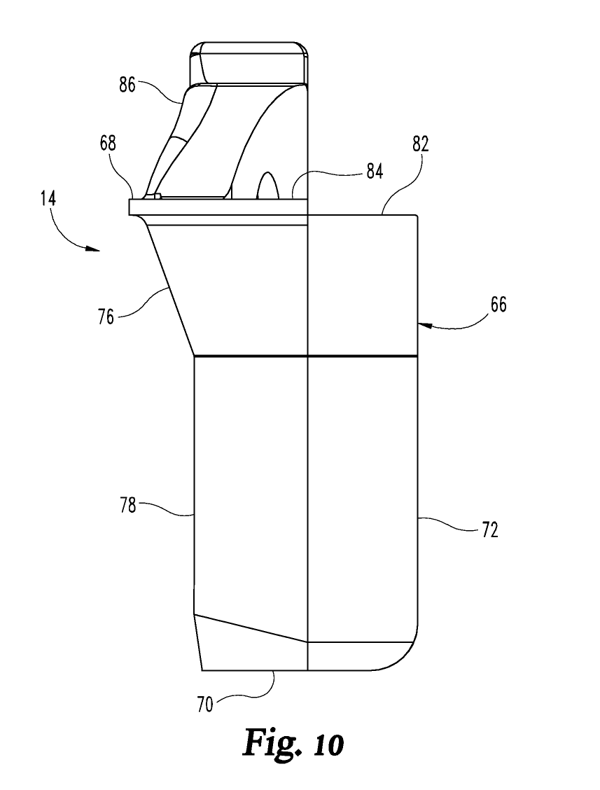

With further reference to FIGS. 8-11, second retractor portion 14 is generally in the form of a retractor blade and includes an elongate body 66 having a sidewall 67 extending between a proximal end 68 and an opposite distal end 70. Sidewall 67 of elongate body 66 further extends between oppositely positioned longitudinal edges 72, 74 which extend between proximal and distal ends 68, 70, and includes a proximal portion 76 obliquely oriented relative to a distal portion 78 at an angle α₂. In one form, angle α₂ is between about 10 and about 30 degrees, although other values for α₂ are contemplated. In other non-illustrated forms, it is contemplated that sidewall 67 can extend linearly between proximal and distal ends 68, 70 without obliquely extending proximal portion 76.

Proximal end 68 includes a rim 80 that extends laterally over proximal portion 76, although forms in which rim 80 is flush with proximal portion 76 are also possible. Rim 80 further includes first and second proximal facing surfaces 82a, 82b that are distally positioned relative to a third proximal facing surface 84. In addition, rim 80 also includes opposite medially facing surfaces 80a, 80b. Extending proximally and laterally from proximal end 68 of elongate body 66 is a second engagement member 86 including a foot 88 (shown in FIGS. 2 and 3 for example) slidably and removably receivable in a corresponding recess of a separation instrument, further details of which will be provided below in connection with FIG. 16. In the illustrated embodiment, foot 88 includes an enlarged outer portion 90 and a smaller cross-section intermediate transition portion 92. First engagement member 86 also includes a hook portion 94 configured to engage with one or more other surgical instruments, non-limiting examples of which will be provided below, or with an external arm that supports retractor assembly 10 while positioned in the patient. For the sake of clarity, it should be appreciated that foot 88 and hook portion 94 are releasably coupled with second engagement member 86 in the illustrated form and have not been shown in FIGS. 8-11. In addition, it should be appreciated that second engagement member 40 may be releasably or non-releasably coupled with elongate body 66, and in some alternative, non-illustrated forms may be absent from second retractor portion 14. While not previously discussed, it should be appreciated that distal end 70 is slightly beveled to facilitate insertion of second retractor portion 14 into an incision, although non-beveled forms for distal end 70 are also contemplated.

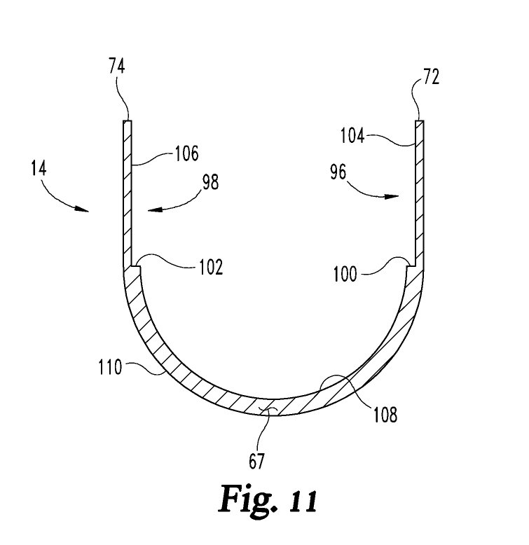

Sidewall 67 of elongate body 66 includes a pair of reduced thickness portions 96, 98 positioned adjacent to longitudinal edges 72, 74, respectively. Similarly, reduced thickness portions 96, 98 define indentations or recessed portions that extend from longitudinal edges 72, 74 to medially facing surfaces 100, 102, respectively. Portions 96, 98 also extend from distal end 70 to proximal facing surface 82. Sidewall 67 of elongate body 66 also includes first and second internal surface portions 104, 106 extending along reduced thickness portions 96, 98, respectively, and a third internal surface portion 108 extending between reduced thickness portions 96, 98. As illustrated in FIG. 11 for example, internal surface portions 104, 106 are outwardly offset away from working channel 16 relative to internal surface portion 108. In contrast, external surface 110 extending between longitudinal edges 72, 74 of elongate body 66 is uninterrupted and substantially smooth, although configurations in which external surface 110 is alternatively arranged are also possible.

While not previously discussed, it should be appreciated that distal portion 78 of sidewall 67 is generally linear in both its longitudinal and lateral aspects along reduced thickness portions 96, 98 such that reduced thickness portions 96, 98 extend substantially parallel to one another along distal portion 78. Moreover, distal portion 78 of sidewall 67 is generally arcuately shaped between reduced thickness portions 96, 98 such that elongate body 66 has a generally u-shaped cross section along distal portion 78 as illustrated in FIG. 11 for example. Moreover, proximal portion 76 of sidewall 67 is generally linear in both its longitudinal and lateral aspects along reduced thickness portions 96, 98, although reduced thickness portions 96, 98 extend transversely to one another along proximal portion 76 given the oblique relationship between proximal and distal portions 76, 78. Moreover, proximal portion 76 of sidewall 67 is generally arcuately shaped between reduced thickness portions 96, 98 such that elongate body 66 also generally has a u-shaped cross section along proximal portion 76. Other cross-sectional shapes are also contemplated for sidewall 67, such as, for example, any open sided polygonal shape or combined curved/polygonal shape, just to provide a few examples.

Second retractor portion 14 can be provided with sufficient rigidity between proximal and distal ends 68, 80 to separate and maintain separation of adjacent tissue when first and second retractor portions 12, 14 are initially inserted and also when the adjacent tissue is retracted by moving first retractor portion 12 and second retractor portion 14 away from one another. For example, second retractor portion 14 can include a thickness between reduced thickness portions 96, 98 which provides sufficient rigidity to resist bending or bowing under the forces exerted on it by the retracted tissue and/or muscle. Also, the generally semicircular or u-shaped cross-section of second retractor portion 14 can be configured to provide a sufficient section modulus or moment of inertia in the direction of movement of second retractor portion 14 to resist bending, bowing and/or deflection forces applied during such movement.

Referring again generally to FIGS. 1-3, first and second retractor portions 12, 14 are positioned in an overlapping arrangement with one another to facilitate insertion of first and second retractor portions 12, 14 into an incision of a patient. More particularly, as illustrated in FIG. 3 for example, reduced thickness portions 50, 52 cooperate with reduced thickness portions 96, 98 such that longitudinal edges 26, 28 are positioned between longitudinal edges 72, 74. Further, in the insertion or unexpanded configuration illustrated in FIGS. 1-3, longitudinal edges 26, 28 of first retractor portion 12 abut against medially facing surfaces 100, 102 of second retractor portion 14, and longitudinal edges 72, 74 of second retractor portion 14 abut against medially facing surfaces 54, 56 of first retractor portion 12. Moreover, this arrangement between first and second retractor portions 12, 14 results in external surface portion 62 of sidewall 19 being positioned adjacent to and flush with external surface 110 of sidewall 67 when retractor assembly 10 is in its insertion or unexpanded configuration. Similarly, when retractor assembly 10 is in this configuration, the outer periphery defined by first and second retractor portions 12, 14 generally extends in a single plane, and is also generally continuous and smooth.

However, it should be appreciated that forms in which longitudinal edges 26, 28 and 72, 74 do not abut against medially facing surfaces 100, 102 and 54, 56, respectively, in the insertion or unexpanded configuration illustrated in FIGS. 1-3 are also contemplated. In addition, medially facing surfaces 34a, 34b of rim 34 of first retraction portion 12 abut against medially facing surfaces 80a, 80b of rim 80 of second retractor portion 14 when retractor assembly 10 is in the insertion or unexpanded configuration, although forms in which medially facing surfaces 345, 346 do not abut against medally facing surfaces 80a, 80b when retractor assembly 10 is in the insertion or unexpanded configuration are also possible. Moreover, first and second retractor portions 12, 14 are further configured to mate with one another such that distal facing surface 38 of rim 34 of first retractor portion 12 is positioned proximally over and slidable along proximal facing surfaces 82a, 82b of second retractor portion 14. Further, in the illustrated form a space extends between first and second engagement members 40, 86 when retractorassembly 10 is in the insertion or unexpanded configuration, although forms in which first and second engagement member 40, 86 are positioned adjacent to one another are also possible.

As indicated above, working channel 16 is formed between first and second retractor portions 12, 14. Working channel 16 extends between and opens at distal ends 22, 70 and proximal ends 20, 68. Moreover, in the insertion configuration illustrated in FIGS. 1-3, working channel 16 is peripherally surrounded or enclosed by first and second retractor portions 12, 14, and has an oblong oval or racetrack-shaped configuration. Stated alternatively, working channel 16 includes a pair of opposite linear portions that are connected on their ends by semi-circular portions. In addition, working channel 16 also generally includes a frusto-conical configuration adjacent proximal ends 12, 14 in order to facilitate greater manipulation of instruments in working channel 16. It is also contemplated that working channel 16 may be provided with alternative shapes and configurations in other non-illustrated forms.

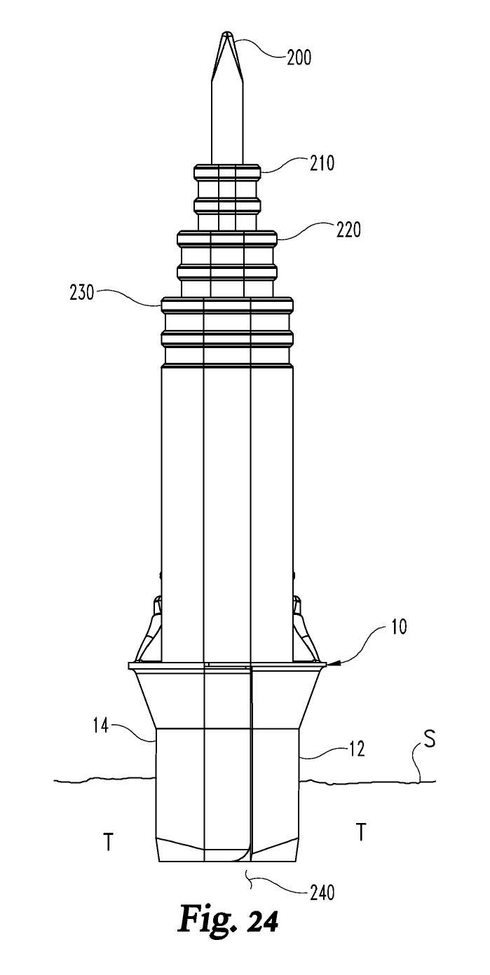

First and second retractor portions 12, 14 are insertable through an incision in skin S and tissue T (FIG. 24) of a patient to provide working channel 16 to a surgical site 240 (FIG. 24) near distal ends 22, 70. It is contemplated that retractor portions 12, 14 are inserted through skin S and tissue T in an insertion configuration for working channel 16, such as shown in FIG. 1. Working channel 16 can have a size in the insertion configuration that allows passage of one or more surgical instruments and/or implants to the surgical site 240 in the patient's body. It may be desirable during surgery to provide greater access to the surgical site 240 in the patient's body beyond the locations provided through working channel 16 in its insertion configuration. Similarly, working channel 16 can be enlarged by separating first retractor portion 12 and second retractor portion 14 along translation axis 112 extending between first and second retractor portions 12, 14. Separation of retractor portions 12, 14 increases the size of working channel 16 from proximal ends 20, 68 to distal ends 22, 70, and can be performed with a separation instrument, one non-limiting embodiment of which will be described below in connection with FIG. 16.

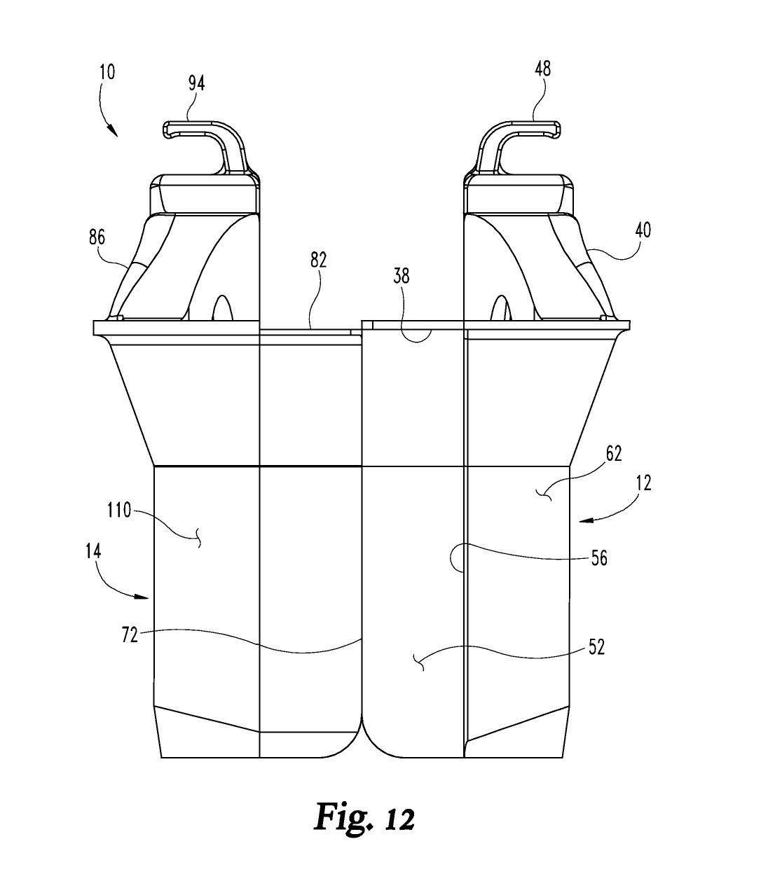

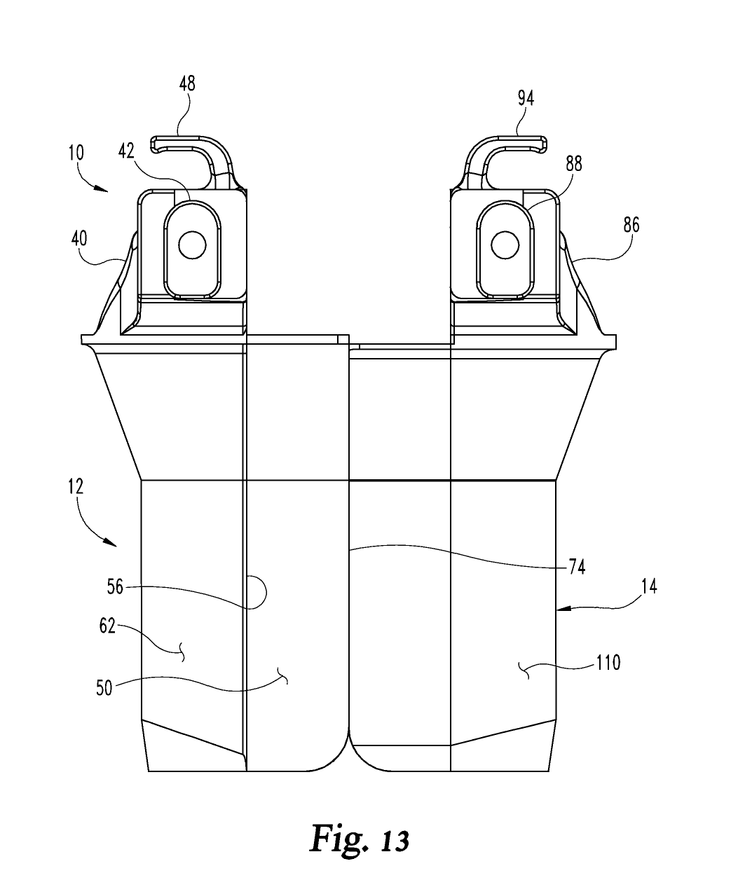

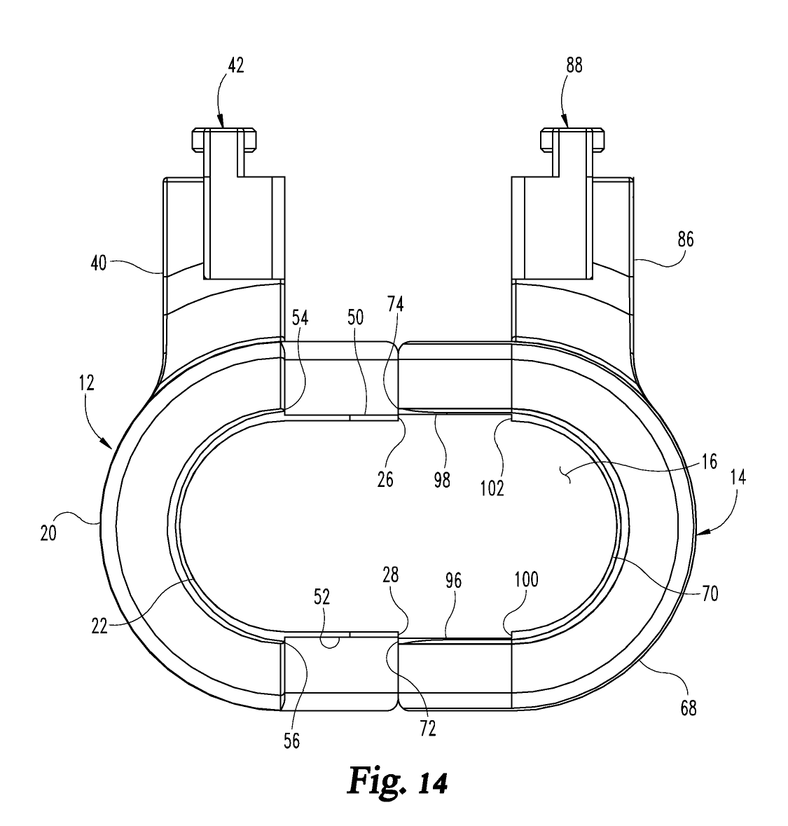

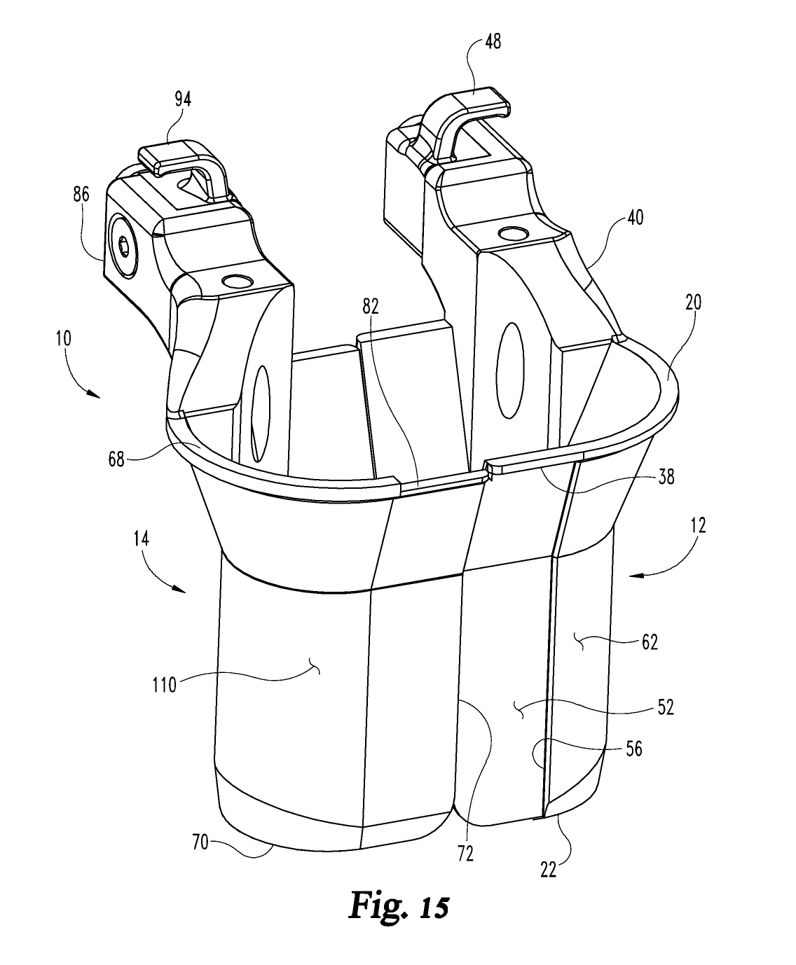

Referring now generally to FIGS. 12-15, retractor assembly 10 is illustrated with working channel 16 enlarged from the insertion configuration illustrated in FIGS. 1-3. More particularly, as first and second retractor portions 12, 14 are laterally displaced from one another along translation axis 112, reduced thickness portions 50, 52 of first retractor portion 12 slide along or near reduced thickness portions 96, 98 of second retractor portion 14. Moreover, first and second retractor portions 12, 14 can be laterally displaced from one another along translation axis until longitudinal edges 26, 28 are positioned adjacent to longitudinal edges 72, 74 as best seen in FIG. 14 for example. As first and second retractor portions 12, 14 are laterally displaced from one another, a space is created between external surface portion 62 of sidewall 19 and external surface 110 of sidewall 67. In the illustrated configuration, working channel 16 remains peripherally surrounded or enclosed by first and second retractor portions 12, 14 following expansion from the insertion configuration illustrated in FIGS. 1-3. As a corollary, retractor portions 12, 14 prevent obstruction of working channel 16 by surrounding skin S and tissue T when working channel 16 is in its insertion configuration and upon its expansion to the configuration illustrated in FIGS. 12-15 for example.

Moreover, it should also be appreciated that first and second retractor portions 12, 14 can be laterally displaced relative to one another to provide working channel 16 with a number of different sizes or configurations between the configurations illustrated in FIGS. 1-3 and FIGS. 12-15 while still peripherally surrounding or enclosing working channel 16. Similarly, working channel 16 can continually be enclosed or peripherally surrounded by first and second retractor portions 12, 14 as first and second retractor portions 12, 14 are moved relative to one another to provide working channel 16 with any number of different sizes or configurations. However, it should also be appreciated that forms in which first and second retractor portions 12, 14 are moved laterally away from one another beyond the configuration illustrated in FIGS. 12-15 are also contemplated.

While not illustrated, it is contemplated that retractor assembly 10 may include arrangements for aligning and releasably coupling first retractor portion 12 and second retractor portion 14 in the insertion configuration. For example, one of retractor portions 12, 14 can include one or more alignment pins which are structured to engage with a corresponding alignment aperture in the other of retractor portions 12, 14. Other arrangements are also contemplated for aligning and releasably coupling first retractor portion 12 and second retractor portion 14 to one another. Examples of such arrangements include dovetail connections, fasteners, threaded coupling members, clamping members, snap rings, compression bands, straps, ball-detent mechanisms, and releasably interlocking cams or tabs, just to name a few possibilities.

In another non-illustrated form, it is contemplated that retractor assembly 10 may be configured such that first and second retractor portions 12, 14 can be pivoted or rotated toward one another about their proximal ends to provide working channel 16 with a tapered configuration that reduces in size from the distal ends of retractor portions 12, 14 through the skin S to the proximal ends of retractor portions 12, 14. A tapered working channel provides the surgeon greater access and increased visualization of the surgical site 240 while minimizing tissue retraction. The tapered working channel 16 also allows greater angulation of instruments and implants placed through working channel 16, more selection in positioning of instruments and implants within working channel 16, and the ability to position instruments and implants adjacent the inner wall surfaces of the separated first and second retractor portions 12, 14, increasing the room available at the surgical site 240 for multiple instruments and for orienting implants.

As indicated above, working channel 16 can be expanded as necessary by moving first and second retractor portions 12, 14 away from one another along translation axis 112. In addition, while not previously discussed, it should also be appreciated that first and second retractor portions 12, 14 can be moved toward one another along translation axis 112 to return working channel 16 to its insertion configuration. One non-limiting embodiment separation instrument 150 for performing these functions in connection with retractor assembly 10 is illustrated in FIG. 16. Separation instrument 150 generally includes a lateral separator operable to linearly move first and second retractor portions 12, 14 relative to one another along axis 112. The lateral separator can be selectively employed by the surgeon during the surgical procedure to adjust the size of working channel 16 and provide the tissue retraction desired for conducting the surgical procedure through working channel 16.

Separation instrument 150 includes a first connection portion 152 movably coupled with a second connection portion 154. As will be discussed in greater detail below, first connection portion 152 is structured to be releasably coupled to first retractor portion 12, and second connection portion 154 is structured to be releasably coupled to second retractor portion 14. In other forms, it is also contemplated that separation instrument 150 may be non-releasably coupled with first and second retractor portions 12, 14. When coupled therewith, first and second connection portions 152, 154 extend away from first and second retractor portions 12, 14 and away from the proximal end opening of working channel 16 to facilitate access to working channel 16 during the surgical procedure. First and second connection portions 152, 154 are operable to move first and second retractor portions 12, 14 toward and away from one another to separate tissue.

First connection portion 152 includes a first extension arm 156 and a coupling arm 158 transversely oriented to and extending from an end of first extension arm 156. A bracket member 160 extends from coupling arm 158, and is engageable by a flexible arm mounted to a surgical table, for example. Second connection portion 154 includes a second extension arm 162 and a housing 164 that extends from an end of second extension arm 154. Housing 164 includes a passage through which coupling arm 158 is movably received. An adjustment mechanism 166 mounted to housing 164 is engageable to coupling arm 158 and operable to translate coupling arm 158 in housing 164 to effect movement of first and second retractor portions 12, 14 toward and away from one another along translation axis 112.

In the illustrated embodiment, coupling arm 158 includes a number of ratchet teeth 168 formed therealong, which are engageable by adjustment mechanism 166. Adjustment mechanism 166 includes a gear wheel 170 with teeth that interdigitate with teeth 168 to effect movement of coupling arm 158 in housing 164 as handle 172 is rotated. A locking mechanism 174 is spring-biased into engagement with teeth 168, and maintains separation of first and second retractor portions 12, 14 when handle 172 is released. Locking mechanism 174 can also be depressed to pivot its engagement end out of engagement with teeth 168 and allow first and second retractor portions 12, 14 to move toward one another.

First and second engagement arms 156, 162 include recesses 176, 178 adapted to receive first and second engagement members 40, 86. Recess 176 includes a keyway opening 180 and a receptacle 182 in communication with opening 180. Receptacle 182 is enlarged relative to opening 180, and is shaped to receive a portion of first engagement member 40 therein. More particularly, foot 42 is slidably and removably received in recess 176 of engagement arm 156 such that intermediate transition portion 46 is received in the intermediate keyway opening 180 and enlarged outer end portion 44 is received in receptacle 182. Similarly, recess 178 includes a keyway opening 184 and a receptacle 186 in communication with opening 184. Receptacle 186 is enlarged relative to opening 184, and is shaped to receive a portion of the second engagement member 86 therein. More particularly, foot 88 is slidably and removably received in recess 178 of engagement arm 162 such that intermediate transition portion 92 is received in the intermediate keyway opening 184 and enlarged outer end portion 90 is received in receptacle 186.

Other configurations for the recesses 176, 178 are also contemplated, including recesses that are enclosed, uniform, or any other suitable configuration to receive at least a portion of an engagement member. Still other embodiments contemplate that engagement arms 156, 162 do not include recesses, but rather are shaped for receipt in or otherwise engage the respective first and second engagement members 40, 86. In one or more other forms, it should be appreciated that engagement members 40, 86 may be engaged to engagement arms 156, 162 of separation instrument 150 by dovetail connections, fasteners, threaded coupling members, clamping members, snap rings, compression bands, straps, ball-detent mechanisms, releasably interlocking cams or tabs, welding, fusing, and/or adhering, just to name a few possibilities, or may also be integrally formed therewith. Other non-limiting separation instruments which may be used with retractor assembly 10 are found in U.S. Pat. No. 7,473,222, the contents of which are incorporated herein by reference in their entirety.

One particular application for retractor assembly 10 is in spinal surgery. It is contemplated that, after insertion of retractor portions 12, 14, they are separated predominantly in one direction to retract muscle and tissue along axis 112 which extends between first and second retractor portions 12, 14. For example, first and second retractor portions 12, 14 of retractor assembly 10 can be primarily or predominantly separable in the direction of the spinal column axis. The muscle tissue adjacent the spine has a fiber orientation that extends generally in the direction of the spinal column axis. The separation of retractor portions 12, 14 of retractor assembly 10 can also separate the muscle tissue along the fibers, thus the amount of separation and the resultant tearing and trauma to the muscle tissue can be minimized. It is also contemplated in other techniques employing retractor assembly 10 that working channel 72 can be enlarged primarily in a direction other than along the spinal column axis or in areas other than spine.

In one example, a method for positioning retractor portions 12, 14 through skin S and tissue T includes making an incision through skin S adjacent the location of a surgical site 240. For example, in spinal surgery, the incision can be made at a vertebral level at a location that provides access to the disc space between adjacent vertebrae or to one or more vertebra through a desired approach. Prior to insertion of retractor portions 12, 14, skin S and tissue T can be sequentially dilated via a dilation instrument set which can include guidewires and/or one or more tissue dilators of increasing cross-sectional size.

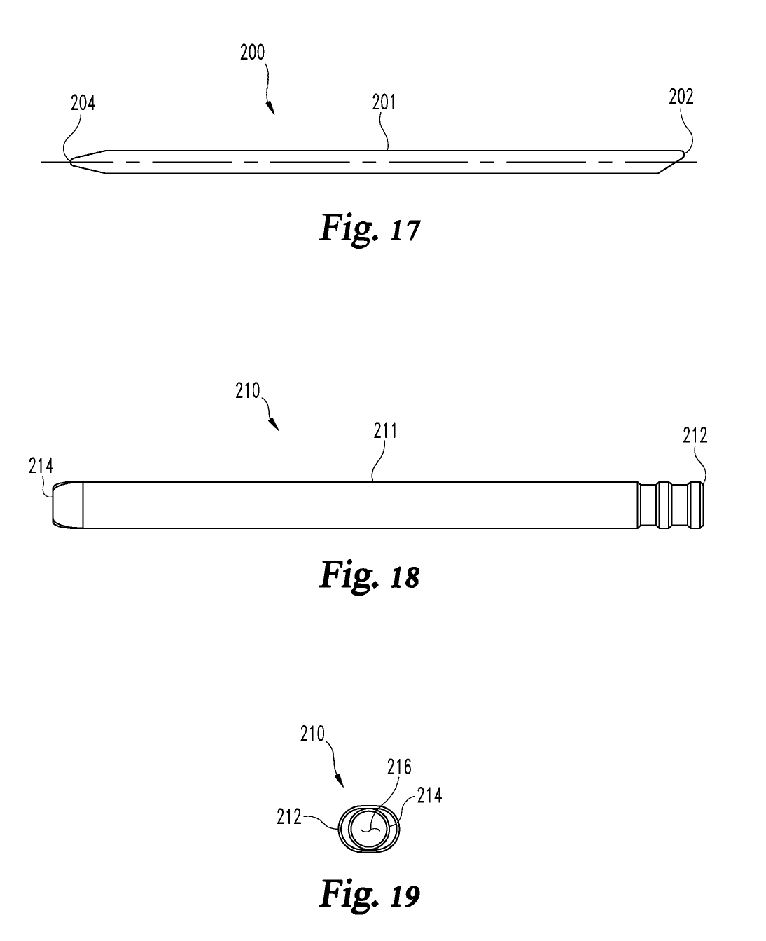

More particularly, with reference to FIGS. 17-23, various dilators of one non-limiting embodiment dilation instrument set are illustrated. For example, in FIG. 17 there is illustrated a first dilator 200 that includes an elongate body 201 extending between a proximal end 202 and an opposite distal end 204. Distal end 204 generally includes a rounded or pointed configuration to facilitate passage of first dilator 200 through skin S and tissue T. In addition, proximal end 202 also includes a partially pointed or rounded configuration that may also facilitate passage of first dilator 200 through skin S and tissue T. Similarly, it should be appreciated that first dilator 200 is configured such that either of proximal end 202 or distal end 204 can be advanced through an incision in skin S and tissue T. While not illustrated in FIG. 17, it should be appreciated that first dilator 200 may also be provided with an elongate bore to facilitate positioning of first dilator 200 over a previously inserted guidewire or k-wire. In addition, in the illustrated embodiment, first dilator 200 generally includes a round or circular cross-section shape, although other variations in the cross-section shape of first dilator 200 are possible.

Referring now to FIGS. 18 and 19, a second dilator 210 includes an elongate body 211 extending between a proximal end 212 and an opposite distal end 214. In the illustrated form, second dilator 210 is shorter than first dilator 200, although forms in which dilators 200, 210 are the same length or dilator 200 is shorter than dilator 210 are also possible. Distal end 214 generally includes a tapered or beveled configuration to facilitate passage of first dilator 210 through skin S and tissue T. As illustrated in FIG. 19, which shows a distal end view of second dilator 210, a longitudinal bore 216 extends through second dilator 210 and opens at proximal and distal ends 212, 214. Bore 216 includes a round or circular cross-section shape generally configured to correspond to the cross-sectional size and shape of first dilator 200 such that second dilator 210 can be inserted over and guided along first dilator 200. As also illustrated in FIG. 19, second dilator 210 generally includes an elongated oval or racetrack outer cross-section shape.

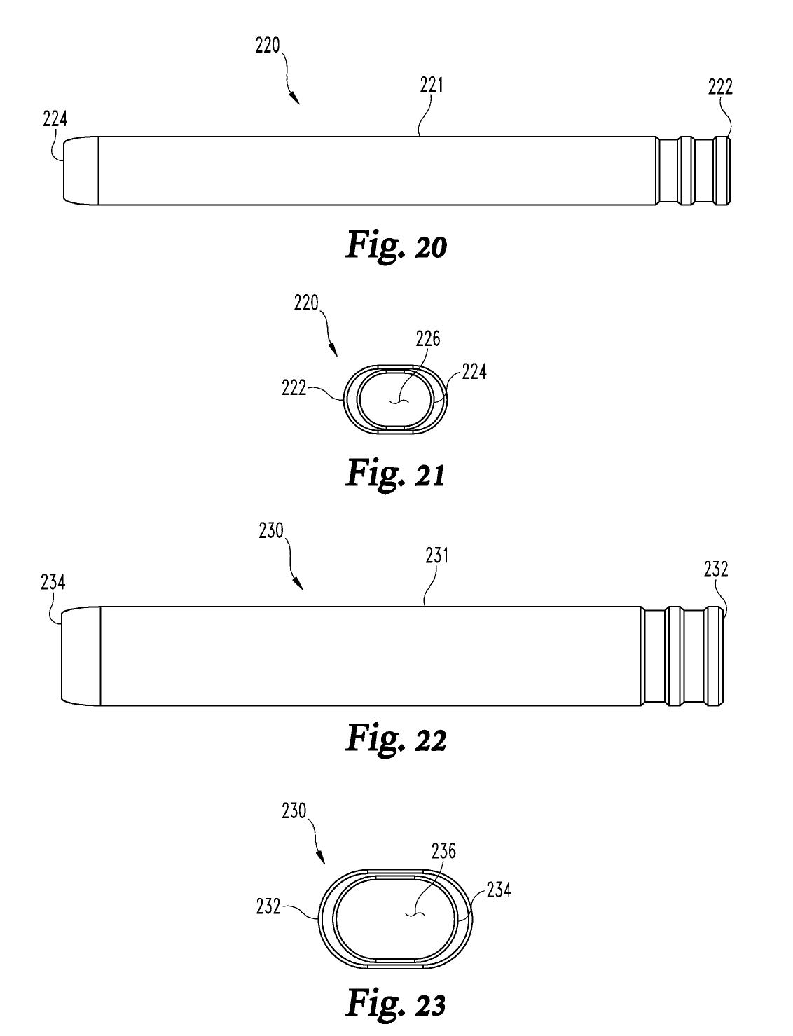

A third dilator 220 is illustrated in FIGS. 20-21 and includes an elongate body 221 extending between a proximal end 222 and an opposite distal end 224. In the illustrated form, third dilator 220 is shorter than second dilator 210, although forms in which dilators 210, 220 are the same length or dilator 210 is shorter than dilator 220 are also possible. Distal end 224 generally includes a tapered or beveled configuration to facilitate passage of third dilator 220 through skin S and tissue T. As illustrated in FIG. 21, which shows a distal end view of third dilator 220, a longitudinal bore 226 extends through third dilator 220 and opens at proximal and distal ends 222, 224. Bore 226 includes an elongated oval or racetrack cross-section shape generally configured to correspond to the outer cross-sectional size and shape of second dilator 210 such that third dilator 220 can be inserted over and guided along second dilator 210. As also illustrated in FIG. 21, third dilator 220 generally includes an elongated oval or racetrack outer cross-section shape.

A fourth dilator 230 is illustrated in FIGS. 22-23 and includes an elongate body 231 extending between a proximal end 232 and an opposite distal end 234. In the illustrated form, fourth dilator 230 is shorter than third dilator 230, although forms in which dilators 220, 230 are the same length or dilator 220 is shorter than dilator 230 are also possible. Distal end 234 generally includes a tapered or beveled configuration to facilitate passage of fourth dilator 230 through skin S and tissue T. As illustrated in FIG. 23, which shows a distal end view of fourth dilator 230, a longitudinal bore 236 extends through fourth dilator 230 and opens at proximal and distal ends 232, 234. Bore 236 includes an elongated oval or racetrack cross-section shape generally configured to correspond to the outer cross-sectional size and shape of third dilator 220 such that fourth dilator 230 can be inserted over and guided along third dilator 220. As also illustrated in FIG. 23, fourth dilator 230 generally includes an elongated oval or racetrack outer cross-sectional shape which is generally configured to correspond in size and shape to working channel 16 in its unexpanded or insertion configuration.

As indicated above, skin S and tissue T can be dilated by sequentially inserting dilators 200, 210, 220, 230 one over another to form a pathway through skin S and tissue T to the surgical site 240 in the patient. In certain forms, one or more dilators in addition to dilators 200, 210, 220, 230 may be used, although forms in which less than all of dilators 200, 210, 220, 230 may be used are also possible. Once the last dilator, such as dilator 230 in the form illustrated in FIG. 24 has been inserted, retractor portions 12, 14 are positioned thereover and guided therealong through skin S and tissue T. While not illustrated in FIG. 24, it should be appreciated that insertion instrument 150 may be coupled with first and second retractor portions 12, 14 either before or after they are inserted through skin S and tissue T. Once first and second retractor portions 12, 14 have been inserted through skin S and tissue T, dilators 200, 210, 220, 230 can be removed to provide access to working channel 16.

For the entire surgery or for certain procedures during the surgery, it may be desired by the surgeon to increase the size of working channel 16 to facilitate access to surgical site 240. First and second retractor portions 12, 14 of retractor assembly 10 can be separated from their insertion configuration to a separated configuration in which working channel 16 is enlarged, as shown in FIGS. 12-15 and discussed in greater detail above.

While not previously discussed, it should be appreciated that viewing instruments can be positioned in or adjacent to working channel 16 to facilitate surgeon viewing of the surgical site 240. For example, an endoscopic viewing element can be mounted on the proximal end of one of retractor portions 12, 14 with a scope portion extending along working channel 16. A microscopic viewing element can be positioned over the proximal end of one of retractor portions 12, 14 for viewing the surgical site 240. Other imaging techniques, such as lateral fluoroscopy, can be used alone or in combination with the endoscopic and microscopic viewing elements. It is further contemplated that other instruments can be mounted on the proximal end of one or both of retractor portions 12, 14, such as nerve root retractors, tissue retractors, forceps, cutter, drills, scrapers, reamers, separators, rongeurs, taps, cauterization instruments, irrigation and/or aspiration instruments, illumination instruments, inserter instruments, and the like for use in surgical procedures through retractor assembly 10 at the surgical site 240. Such viewing instruments and other instruments can be employed with working channel 16 in its initial insertion configuration and/or its enlarged configuration.

In one embodiment, a first retractor portion includes a first elongate body extending between a proximal end and an opposite distal end. The first elongate body further includes a first sidewall including a first pair of reduced thickness portions. A second retractor portion includes a second elongate body extending between a proximal end and an opposite distal end. The second elongate body further includes a second sidewall including a second pair of reduced thickness portions. An adjustable working channel extends between the proximal and distal ends of the first and second elongate bodies of the first and second retractor portions. In addition, the first pair of reduced thickness portions of the first sidewall are configured to overlap with the second pair of reduced thickness portions of the second sidewall.

In another embodiment, a first retractor portion includes a first elongate body extending between a proximal end and an opposite distal end. The first elongate body also includes a first sidewall extending between a first pair of longitudinal edges and including a first external surface positioned between the first longitudinal edges. A second retractor portion includes a second elongate body extending between a proximal end and an opposite distal end. The second elongate body also includes a second sidewall extending between a second pair of longitudinal edges and including a second external surface positioned between the second longitudinal edges. A working channel extends between the proximal and distal ends of the first and second elongate bodies of the first and second retractor portions, and is adjustable upon relative movement of the first and second retractor portions. The working channel includes an unexpanded configuration in which the first longitudinal edges are positioned between the second longitudinal edges and the first and second external surfaces are positioned adjacent to and flush with one another.

In still another embodiment, a method for retracting tissue for percutaneous access to a surgical site in a patient includes providing a retractor assembly including first and second retractor portions and a working channel extending therebetween; positioning the first and second retractor portions in an incision opposite of one another; and expanding the working channel from a first configuration to a second, enlarged configuration by laterally displacing the first and second retractor portions away from one another.

The retractor assemblies, devices, apparatuses, systems and methods described herein also have application with other types of instruments and implants, and may be used in other portions of the body besides the spine. The retractor assemblies, devices, apparatuses, systems and methods described herein may also be used in surgical procedures involving animals, or in demonstrations for training, education, marketing, sales and/or advertising purposes. In addition, the retractor assemblies, devices, apparatuses, systems and methods may also be used on or in connection with a non-living subject such as a cadaver, training aid or model, or in connection with testing of surgical systems, surgical procedures, orthopedic devices and/or apparatus.

Any theory, mechanism of operation, proof, or finding stated herein is meant to further enhance understanding of the present application and is not intended to make the present application in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words such as “a,” “an,” at least one,” at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. A retractor assembly for percutaneous surgery in a patient, comprising:
a first retractor portion including a first elongate body extending between a proximal end and an opposite distal end, said first elongate body further including a first sidewall including a first pair of reduced thickness portions;
a second retractor portion including a second elongate body extending between a proximal end and an opposite distal end, said second elongate body further including a second sidewall including a second pair of reduced thickness portions;
an adjustable working channel extending between said proximal and distal ends of said first and second elongate bodies of said first and second retractor portions; and
wherein said first pair of reduced thickness portions of said first sidewall are configured to overlap with said second pair of reduced thickness portions of said second sidewall.

2. The retractor assembly of claim 1, wherein said first sidewall of said first elongate body extends between a first pair of oppositely positioned longitudinal edges and said second sidewall of said second elongate body extends between a second pair of oppositely positioned longitudinal edges.

3. The retractor assembly of claim 2, wherein said first pair of reduced thickness portions of said first sidewall are positioned adjacent to said first pair of longitudinal edges and said second pair of reduced thickness portions of said second sidewall are positioned adjacent to said second pair of longitudinal edges.

4. The retractor assembly of claim 3, wherein said first pair of reduced thickness portions of said first sidewall define a first pair of indentations, said second pair of reduced thickness portions of said second sidewall define a second pair of indentations, and said first pair of indentations are configured to receive said second pair of longitudinal edges of said second elongate body and said second pair of indentations are configured to receive said first pair of longitudinal edges of said first elongate body.

5. The retractor assembly of claim 4, wherein said first pair of indentations are oriented away from said working channel and said second pair of indentations are oriented toward said working channel.

6. The retractor assembly of claim 1, wherein said first and second retractor portions are movable relative to one another to transition said working channel between a first configuration and a second, enlarged configuration, and said first and second sidewalls of said first and second elongate bodies peripherally surround said working channel in said first and second configurations.

7. The retractor assembly of claim 1, wherein a proximal facing surface on said proximal end of said first elongate body is configured to overlap with a distal facing surface on said proximal end of said second elongate body.

8. The retractor assembly of claim 1, wherein each of said first and second sidewalls includes a proximal portion and a distal portion, said proximal portions extending obliquely relative to said distal portions and providing said working channel with a frusto-conical configuration adjacent to said proximal ends of said first and second elongate bodies.

9. The retractor assembly of claim 1, wherein said first sidewall includes a u-shaped cross-sectional configuration between a first pair of longitudinal edges and said second sidewall includes a u-shaped cross-sectional configuration between a second pair of longitudinal edges.

10. The retractor assembly of claim 1, further comprising:
a first extension extending laterally away from said proximal end of said first elongate body;
a second extension extending laterally away from said proximal end of said second elongate body; and
a separation instrument engageable with said first and second extensions and configured to move said first and second retractor portions relative to one another.

11. A retractor assembly for percutaneous surgery in a patient, comprising:
a first retractor portion including a first elongate body extending between a proximal end and an opposite distal end and including a first sidewall, said first sidewall extending between a first pair of longitudinal edges and including a first external surface positioned between said first longitudinal edges;
a second retractor portion including a second elongate body extending between a proximal end and an opposite distal end and including a second sidewall, said second sidewall extending between a second pair of longitudinal edges and including a second external surface positioned between said second longitudinal edges;
a working channel extending between said proximal and distal ends of said first and second elongate bodies of said first and second retractor portions, said working channel being adjustable upon relative movement of said first and second retractor portions; and
wherein said working channel includes an unexpanded configuration in which said first longitudinal edges are positioned between said second longitudinal edges and said first and second external surfaces are positioned adjacent to and flush with one another.

12. The retractor assembly of claim 11, wherein said working channel is expandable from said unexpanded configuration from said proximal ends to said distal ends of said first and second elongate bodies upon relative movement of said first and second retractor portions laterally away from one another, and a space is created between said first and second external upon expansion of said working channel.

13. The retractor assembly of claim 11, wherein a first portion of said proximal end of said first elongate body is positioned proximally over a first proximal facing surface of said proximal end of said second elongate body when said working channel is in said unexpanded configuration.

14. The retractor assembly of claim 13, wherein said proximal end of said second elongate body includes a second proximal facing surface positioned adjacent to said first proximal facing surface, said first proximal facing surface being distally offset relative to said second proximal facing surface.

15. The retractor assembly of claim 13, wherein said first portion of said proximal end of said first elongate body includes a distal facing surface slidable along said first proximal facing surface of said proximal end of said second elongate body upon relative movement of said first and second retractor portions.

16. The retractor assembly of claim 11, wherein said first sidewall further includes a pair of external surfaces positioned opposite of one another and between said first external surface and said first longitudinal edges, each one of said pair of external surfaces being inwardly offset relative to said first external surface.

17. The retractor assembly of claim 16, wherein said second sidewall includes an internal surface oriented toward said working channel and including a first portion, a second portion and a third portion, said second and third portions being positioned opposite of one another and between said first portion and said second longitudinal edges and each being outwardly offset relative to said first portion.

18. The retractor assembly of claim 11, wherein said first sidewall includes a first pair of linear portions positioned adjacent to said first longitudinal edges and a first arcuate portion extending between and linking said first linear portions, and said second sidewall includes a second pair of linear portions positioned adjacent to said second longitudinal edges and a second arcuate portion extending between and linking said second linear portions.

19. A method for retracting tissue for percutaneous access to a surgical site in a patient, comprising:
providing a retractor assembly according to claim 1;
positioning said first and second retractor portions in an incision opposite of one another; and
expanding said working channel from said first configuration to said second, enlarged configuration by laterally displacing said first and second retractor portions away from one another.

20. The method of claim 19, wherein expanding said working channel further includes peripherally surrounding said working channel with said first and second retractor portions.
